(19) United States
(12) Patent Application Publication
(10) Pub. No.: US 2024/0158853 A1
(43) Pub. Date: May 16, 2024

(54) CHOOSING APPROPRIATE KINSHIP BETWEEN PATIENT AND EGG OR SPERM DONOR FOR MEDICALLY ASSISTED PREGNANCY

(71) Applicant: Marshall Linton Herbert, Largo, FL (US)

(72) Inventor: Marshall Linton Herbert, Largo, FL (US)

(21) Appl. No.: 18/204,757

(22) Filed: Jun. 1, 2023

Related U.S. Application Data
(63) Continuation of application No. 16/679,161, filed on Mar. 23, 2020, now abandoned.

(51) Int. Cl. C12Q 1/6881; G16B 20/40

(52) U.S. Cl. CPC C12Q 1/6881; G16B 20/40; C12Q 2600/154

(57) ABSTRACT

Methods and apparatus to improve results of assisted fertility by selecting a donor who is optimally related to the recipient according to genealogy, adjusting for known history of ancestral consanguinity, and folic acid exposure. The selected donor might be third or fourth cousin.

### CROSS REFERENCE TO RELATED APPLICATIONS

This application is a continuation of copending application Ser. No. 16/679,161, filed Mar. 23, 2020, which is incorporated fully herein as if set forth verbatim for all purposes.

### STATEMENT OF FEDERALLY SPONSORED RESEARCH OR DEVELOPMENT

Not applicable. CL REFERENCE TO A SEQUENCE LISTING, TABLE, OR A COMPUTER PROGRAM LISTING APPENDIX SUBMITTED ON A COMPLETED DISC AND AN INCORPORATION BY REFERENCE OF THE MATERIAL ON THE COMPACT DISC THE TOTAL NUMBER OF COMPACT DISC INCLUDING DUPLICATES AND THE FILES ON EACH COMPACT DISC SHALL BE SPECIFIED

Not applicable.

### BACKGROUND OF THE INVENTION

1 Sibly p 2
2 Iceland p 4
3 Denmark p 5
4 Mechanism of the Effect of Kinship on Fertility p 6
5 Folic acid effect p 8

### Sibly

A team led by a man named Richard Sibly wrote “On the Regulation of Populations of Mammals, Birds, Fish and Insects,” Richard M. Sibly, Daniel Barker, Michael C., Denham, Jim Hope and Mark Pagel Science vol. 309 Jul. 22, 2005 page 609. They collected every paper published before 2005 that was about serial field counts of wild animals. If you went into a field periodically and counted rabbits and published your results your paper was analyzed unless the population went extinct. In all, they found 1,700 studies they thought suitable and discussed what they found.

Sibly did not publish an overall diagram, but he looked at the collection and gave a typical example. It looked roughly like this:

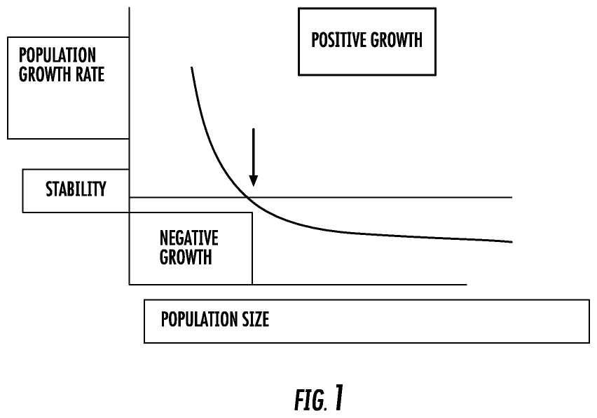

### FIG. 1

The horizontal axis is the animals counted. The vertical axis is the population growth rate. The cause of the growth rate is difference in fertility.

The line is incomplete. There is no evidence shown for inbreeding depression. The line far toward the left should be falling very steeply. We agree that inbreeding depression happens. Sibly says he did not observe it in the wild, but he excluded from analysis populations that went extinct. So, I think it more than fair to say that there is an undemonstrated falloff in fertility at very low population sizes.

The important point is indicated with a red arrow. Here there is neither positive nor negative growth. For humans that is ideal. Opinions may vary on just how many people we want on the planet, but there can be no disagreement on the right growth rate. Negative growth eventually leads to extinction. Positive growth eventually leads to the kind of environmental and resource exhaustion that we have been warned of so much.

Crucially, at smaller population sizes than the zero growth point the fertility rises rapidly. At larger sizes, the fertility falls slowly. This is what we need to see. The curve stabilizes the population size at some moderate value. So long as the displacement from the zero point is not too great the population will return to that point.

If it were possible to divide human kind into a number of mutually exclusive societies, each the size of the population at this point, and if they were to reach equilibrium then we would have a stable world population. Just what that population size should be is not agreed on. It might be possible to work it out from the Icelandic genealogies such as we shall be looking at next. How it could be set up in a way that left everybody happy or even whether there will ever be the will to try are questions that are as baffling as they are important.

### Iceland

We have seen the Sibly curve, which is supported by massive amounts of data, which shows the mechanism in effect, stabilizing populations of mammals, birds, fish and insects. It would be understandable for a prudent person so say, “Ah, but those are all dumb animals. We humans choose whether to reproduce.” And to a degree we can, but we need to look at some human data.

A team led by a man named Helgason looked at data from the enormous Icelandic genealogy. Then he compared the kinship of couples with how many children they had. This is what he got:

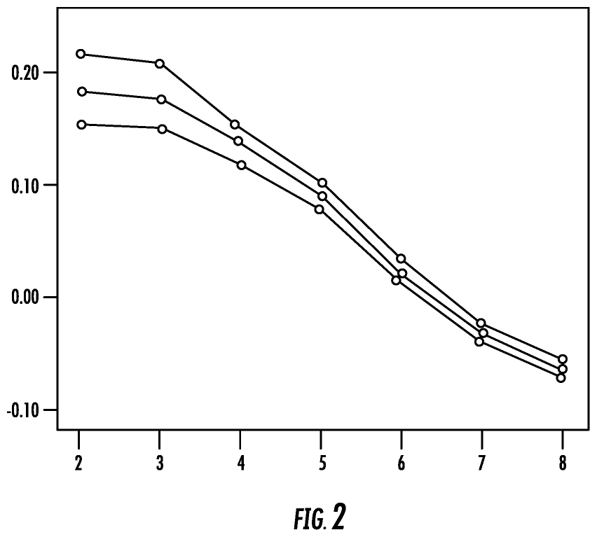

### FIG. 2

“An Association between Kinship and Fertility of Human couples,” Helgeson et al. Science vol. 319 Feb. 8, 2004 page 813.

They chose a large number of couples that had had all the children that could be expected. The researchers recorded the number of children each pair had. They calculated the kinship of the couples by going back ten generations and counting how many ancestors they shared in that generation. Then they calculated kinship in terms of say first cousin if they shared a quarter of the possible number of ancestors, second cousin if they shared an eighth and so forth. Obviously, this is not the way we ordinarily recon kinship. I'm sure very few can name all their ancestors for 10 generations back. Then they lumped the data so that every couple between say third cousin and fourth cousin was labeled “fourth cousin or closer.” Then they took the average number of children in each fraction and “normalized” it, which means they compared it with the average across the country for that year. Thus, zero means the same degree of fertility as the Icelandic average.

We can immediately recognize the Sibly curve in the middle and right end of the Iceland curve. Do not think for a second that humans are exempt from biology. The effect is not enormous, going from roughly 10% below to 20% above the national mean, but the effect is clearly not trivial.

Now look at the error bars. They are very tight. Those are ninety percent confidence limits. There cannot be much else determining fertility. Any factor other than kinship of the couple has to squeeze into that little interval. That includes choice. Look at “eighth cousins or closer.” That is the most distant relationship they examine. Ninth cousins are vanishingly rare or totally absent. That alone should give a prudent person warning.

Helgason's team went further and looked at the next generation, the grandchildren, but we shall not examine this, having already established the Sibly curve.

### Denmark

The next data set comes from Denmark. Rodrigo Labouriau and Antonio Amorim compared fertility in Denmark with something they call “marital radius” (how far apart a couple were born). This is what they found:

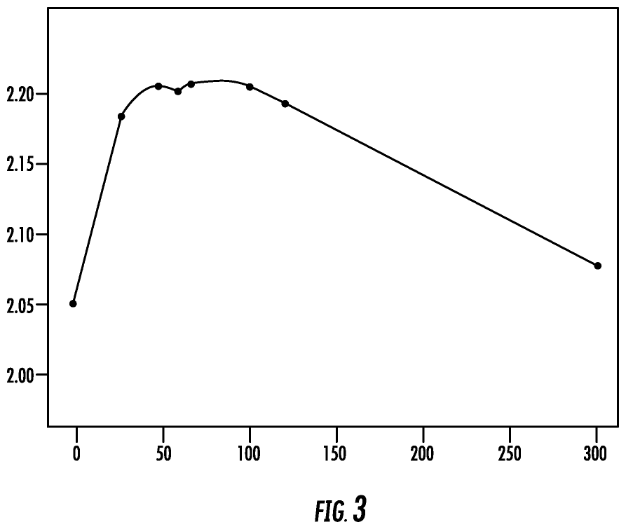

### FIG. 3

Comment on “An Association between Kinship and Fertility of Human Couples,” Rodrigo Labouriau and Antonio Amorim, SCIENCE vol. 322 no. 5908 Dec. 12, 2008 page 1634. For the whole story also see Human Fertility Increases with marital radius. Rodrigo Labouriau and Antonio Amorim. GENETICS volume 178 January 2008 page 603.

It looks like the Sibly curve again. At marital radius fifty kilometers or less we see the inbreeding depression we knew all along had to be there.

At marital radius one hundred kilometers or greater there is a decrease in fertility. It looks like a linear fall, since the team chose to graph population against a line, rather than against an area.

As for choice, the team specifically compared education and income with fertility. They found that after you allow for kinship as measured by town size and marital radius, there was no effect of education OR income on number of children.

### Mechanism of the Effect of Kinship on Fertility

I did an experiment with fruit flies: Fluctuation of fertility with number in a real insect population and a virtual population M. L. Herbert and M. G. Lewis African Entomology 21(1): 119-125 (2013).

The paper by Sibly showed that the mechanism, and he explicitly stated that there had to be a mechanism, as seen in mammals, birds, fish and insects. Of these, insects are so far down the food chain so-to-speak that just about nobody cares much about how you abuse them. I hasten to say that I never planned to and never did deliberately abuse my insects. How they felt about it all they never said.

The most popular insect for laboratory studies is of course the fruit fly, Drosophila melanogaster. They can be purchased cheaply; I got mine from North Carolina Biological Supply. My initial plan was to cross together flies that had been unrelated for a long time. The pleasant technologist I spoke with assured me that all the lines they kept had been there longer than the three years she had worked there. I bought sixteen lines of flies, one wild type and fifteen more each with a specific mutation, so the lines were unrelated for many generations. Standard fruit fly husbandry dictates you start a new line with four males and four females so each time I crossed them I would take four males from one line and four virgin females from another. Calling the lines “a” through “p,” I crossed a with b to get ab, b with c to get bc and so forth until I crossed p with a to get pa. Then I crossed ab with cd, bc with de and so forth, so every generation I kept 16 lines going. Eventually I had lines abcdefghijklmnop, bcdefghijklmnopa and so on for 16 lines, each descended from all of the original 16.

It wasn't so straightforward. Lines would die out, particularly after the first few generations, and my technique was not perfect. Sometimes a male would find its way into a vial of virgin females. I would have to go back and get vials that had gone according to plan.

My four generations never gave me complete infertility. I do not know what would have happened had I been technically able to carry it all out for five or six generations.

I released the flies in a large cage with plenty of feed formula in bottles. Each day I would add four new bottles and remove the four oldest, which had been in there for 28 days. I made daily counts at two measured windows and summed them at two-week intervals. The result was a pattern of damped oscillation of the population numbers. This is unsurprising when one accepts that the Sibly curve is a general law.

### Folic Acid Effect

The speed and predictability of the changes in population size indicate that this is not a matter of genes—mutation and selection—and so the immediate mechanism must be epigenetic, such a methylation of critical parts of chromosomes. Having found the weight of evidence to suggest that something like methylation drives fertility in fruit flies, we must conclude that the statement, “Only kinship determines fertility, minus rare disaster,” is incomplete. One must add, “And something like methylation has an effect.”

Pursuing the issue further, I wondered if folic acid, which potentiates methylation, would have an effect? In an earlier study, a cocktail containing folic acid seemed to have a significant effect over an entire order of magnitude of dilutions, overwhelming the damped oscillation pattern. There is only one concentration of folic acid that is of the greatest importance. Women who are pregnant or liable to become pregnant are advised to take folic acid regularly. Received wisdom is that since folic acid is water soluble, it does not accumulate in the body so large doses are given with impunity. Now folic acid is not itself a natural food, but is thought to substitute for “folate” compounds, three micrograms of folic acid being the equivalent of five micrograms of folate. That dose of folate is about what is found in an apple, if eaten seeds and all. So, three micrograms of folic acid, or one ripe apple daily, should be a pretty good amount even if the rest of the diet is lacking.

“Should be” is not “is,” and what I am about to say must not be taken as advice but as an inducement to further study. Folate is a necessary vitamin. Lack can lead to a lethal anemia, since it is necessary for DNA replication, which is necessary for cell division. Moreover, a lack of folic acid is correlated with a condition called a neural tube defect in a developing pregnancy. The condition can range in severity from an almost trivial sinus tract in the small of the back to a devastating neurological malformation. The condition can be prevented by folate about half the time. Current practice is to give a woman hundreds of micrograms of folic acid a day. Please do not eat a hundred apples a day seeds and all; that would include enough cyanide to kill you even though cyanide is nicely soluble in water.

The pressing question is whether the current recommended dose of folic acid is safe. As best as I could, I prepared two feeding formulae for the flies. One was the standard recommended by the supplier. The other was mixed not in straight water, but in water that had been given folic acid. The amount was chosen such that the final food would have the same amount of folic acid per calorie as a woman eating a good 1,800 calorie diet, supplements and all would get.

My routine was to add four males and four females to a vial and follow them for two weeks. After about 11- or 12-days fresh flies would begin to emerge. As soon as they did, I would shake them out into a vial to be kept giving them normal food. There were three control vials and three fortified.

At the end of two weeks, all the flies that had emerged from all the control vials and all that had emerged from the treated vials were added together and are reported below. I must mention that shaking the vials out requires a bit of a light touch. Shake too little and most of the flies remain in their vial. Shake too hard and the food may drop out as well, drowning a lot of the flies and wrecking the experiment. Good luck. I ran the flies, all six vials, in the control medium for a couple generations and then started adding the spiked water to half of them. This is what I got.

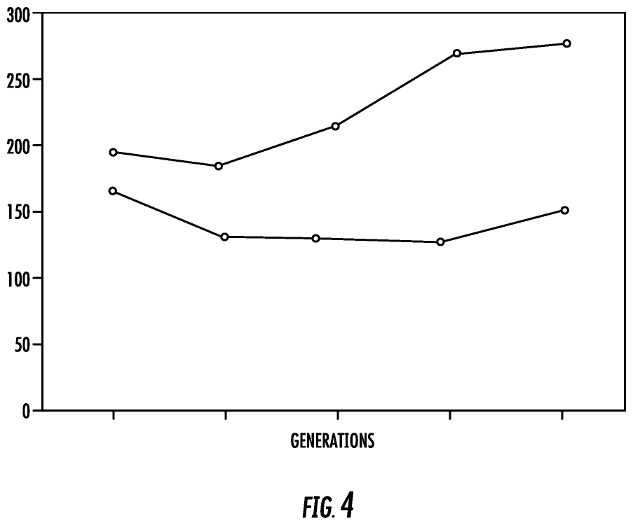

### FIG. 4

The horizontal axis is five successive generations of flies. The vertical axis is the sum of daily counts.

It seems to me that the folic acid reduces fertility. Please, somebody with a better hand, better resources and access to peers give t is a try. Failing that, any youth could do it as a high school science project. You don't really need a dissecting microscope. There are cheaper ways to magnify the flies and be sure of the sex. It may be that mammals have a way of dealing with high folic acid levels. This would be good to know. So far, I do not see any effect of high folic acid levels outside the lab. If folic acid depresses fertility in mammals, assuming they have no defense, then this would shift the Sibly curve to the right and the highest fertility match would be closer kin than suggested by the Iceland study in isolation.

### BRIEF SUMMARY OF THE INVENTION

Using best contemporary medical practice—which of course changes over time—it is possible to obtain medically assisted pregnancy. The success rate is low enough so that there is significant room for improvement. It has been established that fertility and kinship are related in such a way that there is an optimal degree of kinship between a couple, either for achieving some goal such as maximizing fertility or stabilizing a population in the long run. What could be done is to coopt the mechanism that links kinship and fertility by selecting sperm or egg donors of a specific degree of kinship with the recipient. This deliberate use of the mechanism with rational choice of kinship in the context of assisted pregnancy is what is claimed.

### DETAILED DESCRIPTION OF THE INVENTION

Ordinarily a human life begins when a man's sperm and a woman's ovum combine in the supportive environment of her reproductive tract, and generally for the strictly biological part of the process, the two need no help from anybody else. Sometimes, even if all social, emotional, and psychological issues are supportive, the two are unable to produce a healthy baby, and medical assistance is sought, which artificially combines sperm and ovum and sometimes the nucleus of one ovum and the cytoplasm of another, but for purposes of this specification we consider the ovum to be the provider of the nucleus from the woman. Pregnancy can be achieved by such artificial means by having another man provide the sperm or another woman provide an ovum or both.

A study done in Iceland mentioned in the “background” showed that the variation of fertility from one couple to another depends on their kinship and kinship among their ancestors. This is supported by reasoning from population size and fertility in wild animals, the relationship of kinship and fertility in Iceland and Denmark and lab studies with fruit flies. The bulk of the evidence suggests that there are at least two mechanisms of reduced fertility affecting the relationship between kinship and fertility, one mechanism being pre-zygotic and one post-zygotic, both being mediated by methylation of sites on chromosomes and how the methylation patterns are related from certain parts of the genome compared with other specific parts.

Pre-zygotic infertility can be bypassed during assisted fertilization by direct injection of the sperm into the ovum. Post-zygotic infertility at the present level of knowledge can only be controlled by selecting a sperm donor or ovum donor that is near enough kin to the recipient so that the methylation patterns have not had enough generations to change too much to permit a normal pregnancy to proceed.

The invention claimed is to coopt the normal mechanism relating methylation pattern with fertility so as to optimize fertility or achieve some goal, such as the greatest chance of a child, the greatest chance of a grandchild or the establishment of a community in which, barring immigration and emigration, will regulate itself by natural means at a constant size, which appears likely to be something like one or two hundred nuclear families.

A first step would be to identify a suitable cousin who is agreeable to volunteer to be the donor, probably a third or fourth cousin relative to one of the couples seeking a baby. The Iceland study suggests that nearer kin should produce a higher likelihood of a child than the degree of kinship with the highest probability of a grandchild. If there is a large amount of consanguinity among the ancestors of the donor and recipient, the optimal distance as established by genealogy alone would increase. Conversely if there has been high exposure to folic acid, which is known to enhance methylation and thus methylation pattern change, then the optimal degree of separation estimated from genealogy would tend to decrease. Although methylation is generally a natural process, coopting the mechanism to achieve a rational goal means it is effectively a machine, as is a lever or inclined plane.

### SEQUENCE LISTING

Not applicable.

What is claimed is:

1. Improving results of assisted fertility by selecting a donor who is optimally related to the recipient according to genealogy, adjusting for known history of ancestral consan guinity and folic acid exposure. Initially third or fourth
cousin might be the starting place.

2. The same number one plus verifying the kinship with genetic studies.

3. As information and knowledge accumulate, and the actual sites of DNA methylation that control fertility are learned, using that information as well to refine choice of donor.

4. If it is found out how to alter methylation patterns for the better and it can be done safely, that obviously could be done as well.
